(19) United States
(12) Patent Application Publication
(10) Pub. No.: US 2006/0013893 A1
(43) Pub. Date: Jan. 19, 2006

(54) Bisphosphonates inorganic carriers

(76) Inventor: Richard F. Stockel, Bridgewater, NJ (US)

Correspondence Address: RICHARD F. STOCKEL, 475 ROLLING HILLS ROAD, BRIDGEWATER, NJ, 08807, US

(21) Appl. No.: 11/157,003

(22) Filed: Jun. 20, 2005

Related U.S. Application Data
(60) Provisional application No. 60/589,221, filed on Jul. 19, 2004.

(51) Int. Cl. A61K 31/675; A61K 33/42

(52) U.S. Cl. 424/601; 514/89; 514/102

(57) ABSTRACT

Intercalated hydrotalcite-bisphosphonate compositions have improved bioavailability, less irritation in the GI tract and a more constant controlled delivery rate of the active component. These hybrid organic drug—inorganic carrier are useful to build-up skeleton bone and to incorporate into a dentifrice to strengthen the hydroxyapatite mineral on teeth. Added advantages in using hydrotalcite are that it is an excellent anti acid ingredient and certain anionic forms of analgesics vitamins, and pro-vitamins can be intercalated into the clay providing synergy to the bisphosphonate therapy. Other bioactive molecules in their anionic form can also be included, e.g., certain hormones, and nutritional supplements to name a few classes.

### BACKGROUND OF INVENTION

Considerable research efforts have been conducted on oral sustained and controlled release drug delivery systems. Special attention has been given to regulate the rate of drug release by means of monolithic devices, where the drug is dispersed or included in an inert matrix. One of many such systems involves the intercalation of a drug in a lamellar host lattice. Several natural and synthetic clays having either cationic or anionic replaceable ions by simple exchange methods are ideal carriers for charged drug molecules.

For example bentonite can exchange with cationic drugs, and hydrotalcite can exchange with anionic drugs

Bentonite and hydrotalcite are only two examples of clays that form intercalated drug delivery systems.

This invention deals with new and novel compositions and formulations of bisphosphonates with hydrotalcite-like clays resulting in lamellar host lattice intercalated drug delivery systems. Bisphosphonates are a family of bioactive drugs that have been used in the management of disorders of calcium and bone metabolism for the past three decades. The therapeutic potential of bisphosphonates is due to their patent inhibition of osteoclast mediated bone resorption. By varying the substuents on the methylene group located between two phosphonic acid groups many different derivatives have been synthesized, and several have been commercialized. Neutral methylene functionalities include; hydroxy, alkyl, aryl; basic functionalities include amino, heterocyclic; acid functionalities include carboxylic acid, sulfonic acid.

### SUMMARY OF THE INVENTION

This invention is based on intercalation chemistry involving bisphosphonates and hydrotalcite-like anionic clays. The various layered materials useful as the host for the anionically charged bisphosphonates are collectively known as layered double hydroxides. These are part of a large class of materials closely related to the mineral hydrotalcite and represented by the general formula [M²⁺₁₋ₓM³⁺ₓ(OH)₂]Aⁿ_{x/n}.yH₂O; or [M¹⁺M³⁺₂(OH)₆]Aⁿ⁻_{x/n}.yH₂O; where M¹=Li, Na, K, Rb or Cs; M²⁺=Ca, Mg, Mn, Co, Ni, Cu, Zn, and Cd; and M⁺³=Cr, Fe, Al, Ga, In, Mo, A maybe an organic anion, e.g., bioactive bisphosphonate, or an inorganic anion such as PO₄⁻³, NO₃⁻; Cl⁻, Br⁻; I⁻, ClO⁻₄, SO₄⁻², or CO₃⁻², the value of x is usually between 0.2 and 0.33 related class of materials and having in general, a very similar intercalation chemistry are the hydroxy double salts of the general formula; (M²⁺)₅ (OH)₈(Aⁿ⁻ⁿ)_{2/n}yH₂O or (M⁺²)₂(OH)₃.(Aⁿ⁻)_{1/n}.yH₂O; wherein M is typically Zn, Cu, or Ni. These remarkable materials are closely related to the mineral brucite, Mg (OH)₂, which has a layered structure consisting of sheets of edge-sharing Mg (OH)₆ octahedra. The hydrotalcite structure is derived from brucite by substitution onto the cation sublattice, and the subsequent uptake of the anion into the interlayer region to preserve charge neutrality.

Hydrotalcite is effective against the treatment of ulcers. It is believed that the high anti-peptic activity of hydrotalcite may be due to its ability to absorb the negatively charged pepsin onto its positively charged surface. Hydrotalcite is also capable of buffering the pH of the stomach at about 4 for a long time. Thus gastric juice is maintained at a pH that is neither too acidic for high pepsin activity nor too alkaline to trigger the acid rebound. Moreover, the rate of hydrochloric acid neutralization by hydrotalcite increases with pepsin concentration.

A comparison of different antacid compounds (at doses with at least comparable neutralizing capacity) also demonstrated that the hydrotalcite displays the lowest intestinal absorption and does not increase the level of aluminum in the serum (van der Voet et al., Clinical Toxicology, 24 (1986-87) p. 545).

The dissolution of any Al⁺³ ions is negligible as reported by van der Voet in the serum of a patient taking hydrotalcite formulations. If need be, the exchangeable carbonate can be replaced by phosphate, or sodium phosphate can be part of the total dosage, and any Al⁺³ ions will react with phosphate to yield a very water insoluble aluminum phosphate.

A unique aspect of our invention involves the anionic exchange capability of hydrotalcite, whereby certain non-steroidal anti-inflammatory drugs or NSAID's, which are able to form an anion can be administered along with a bisphosphonate simultaneously.

Examples of some, not all inclusive, NSAID's are aspirin, ibuprofren, diclofenac, diflunisal, etodolac, fenoprofen, flurbiprofen, ibuprofen, indomethacin, ketoprofan, ketofolac, mefenamic acid, naproxen, oxaprozin, piroxicam, salsalate, sulindac, tolmetin and the like.

Other bioactive molecules, capable of forming an exchangeable anion with the hydrotalcite anion, are vitamins C and E, thereby resulting in synergy. Both vitamins can be intercalated with hydrotalcite and hydrotalcite like clays. Vitamins C and E are known to benefit the maintenance of healthy bone structure and growth. For example vitamin C functions to help maintain collagen, a protein necessary for forming skin, ligaments, teeth, and bones. Vitamin E derivatives have beneficial effects on bone calcium in adrenalectomized animal studies (S. Ima-Nerwana, J. Med. Food, 2004, Spring 7(1)45-51).

It is also known that patients with peptic disorders e.g., peptic ulcers generally have low leucocyte levels of ascorbic acid. This is also true for patients with gastroduodenal problems. Thus the availability of Vitamin C would help to alleviate these conditions.

Amino carboxylic and amino phosphonic chelating agents like EDTA can also be incorporated as an anion.

Obviously, several biological actives could be administered using the teachings of this invention, provided they have a therapeutic value.

Any bioactive bisphosphonate useful for the treatment of osteoporosis can be affixed to hydrotalcite-like anionic exchange clay via a sodium salt of the drugs phosphonate function. The resulting phosphonate anion can exchange with the anions in between the lamellar lattice. Obviously other water-soluble anionic salts are also operative according to the teachings of this invention. Suitable bisphosphonates are pamidromate, ibandronic acid, ibandronate, risedronate, cimadronate, clodronate, etidronic acid, neridronate, olpadronate, piridronate, tiludronate, zolendronate, icadronate, and pharmaceutically acceptable salts thereof.

The anionic exchangeable clays of this invention, most preferred are hydrotalcite-like clays, which has been described in the specifications of this application. The exchangeable anion can be chloride, carbonate, sulfate, nitrate or other non-coordinated anions, which are easily displaced by bisphosphonate anions or the free acid.

While the hydrotalcite-like clays can vary within theoretical limits the composition of the synthetic material used in these experiments had the formula: Mg0.67 Al 0.33 (OH)₂ Cl0.33·0.6H₂O (ref. Clays Clay Miner, 1983; 31; 305-311 and references cited in article). The ion exchange capacity is 3.9 meq/gram. It is understood by those skilled in the fields of chemistry that other compositions, as described in this invention, can be successfully substituted to achieve teachings of this technology.

With respect to the drug release rates, fortunately there are several modifications, which can be utilized to extent the delivery of the bisphosphonate to assure better bioavailability beyond the drug-hydrotalcite composition. One such modification is to coat said composition with a water-soluble or hydrocolloid polymeric substance, e.g., cellulose, hydroxyalkylcellulose, corn starch, gum arabic, alginate, polyvinyl alcohol, carboxymethylenecellulose, polyvinyl pyrollidione, acrylic resins, polyethylene glycol waxes, carrageenan cellulose acetate phthalate and the like. These specific examples are just a few known in the pharmaceutical industry useful for enteric coatings.

A second approach to extent the release of the bisphosphonate-hydrotalcite is achieved by co-administering a mixture of unloaded hydrotalcite with the drug-clay composition. In general the weight ratio of drug-clay to clay was 1:1.1 to 1:2 w/w. By changing variables such as loadings, particle size and ratios it is possible to further optimize the rate of release of the bisphosphonate. Enteric coatings are also useful in the final formulation in the administration of the drug.

Yet another option for controlling the release can be used to carry out the teachings of this invention involves the physical admixture of a bisphosphonate and a hydrotalcite clay. As with the other methods of changing the release of the bisphosphonate enteric coatings are an option.

The bisphosphates-hydrotalcite clays of this invention are preferably those for internal administration, e.g., unit dosage forms such as oral, vaginal and rectal formulations, e.g., tablets, capsules, syrups, drops or suppositories. It is also possible to use stable slurries of these formulations that are used for injections.

The novel pharmaceutical compositions of the present invention are prepared in a manner known per se, for example by conventional mixing, granulating, confectioning, dissolving or lyophilizing method.

Suitable carriers are in particular fillers such as sugar, for example lactose, saccharose, mannitol, or sorbitol, cellulose or derivatives thereof and/or calcium phosphates and also binders such as starches and/or water-soluble polymers or hydrocolloids. The latter can also be used as an outer coating to achieve various degrees of control release. Other adjuncts useful as glidants and lubricants like silica, talc, stearic acid/derivatives thereof, are also used for the formulation of these drugs.

Gelatin capsules are yet another form of delivery. Plasticizers like glycerol, propylene glycol, low molecular weight polyethylene glycols or sorbitol are useful in the preparation of said capsules.

The teachings of this invention offer several advantages over the prior art in terms of administrating an effective amount of a bioactive bisphosphonate to manage disorders of calcium and bone metabolism. The therapeutic potential of bioactive bisphosphonates is due to their potent inhibition of osteoclast mediated bone resorption. The dosage forms prohibit the exposure of the bisphosphonates to the epithelial and mucosal tissue of the buccal cavity, pharynx, esophagus, and stomach and thereby protects said tissues from erosion, ulceration or other like irritation. According, the bisphosphonates-hydrotalcite dosage forms effect the delivery to the lower intestinal tract of said human or other mammal of a safe and effective amount of the bisphosphonate drugs, and substantially alleviate the esophagitis or esophageal irritation which often accompanies the oral administration of bisphosphonates active ingredients.

Another advantage of this invention is the prolonged release of the bisphosphonate from the hydrotalcite, or the hydrotalcite matrix tablet whereby increasing they bioavailability of the active drug.

Toxicity is also another advantage in the dosage forms of this invention by virtue of the inert and non-toxic usage of hydrotalcite in the delivery of a bisphosphonate. Hydrotalcites are not metabolized and it is readily removed from the body.

Another advantage of the dosage forms of this invention is the ability of the drug-clay composition to mask the taste of bioactive bisphosphonates. This complex passes unchanged through the gastric system into the intestinal tract.

Experimental

Well crystallized Mg0.67 Al0.33 (OH)₂ Cl0.33.0.4 H₂O was synthesized as described in Eur. J. Inorg. Chem. 1998; 10:1439-1446. Alendronate sodium trihydrate and risedronate sodium was purchased from a Chinese source.

Intercalation of the Bioactive Bisphonates with Hydrotalcite-Like Clay with Chloride Exchangeable Anions.

Intercalation reactions were performed by equilibrating hydrotalcite and the bisphosphonate in a aqueous ethanol mixture (50:50_{v/v}) at 60° C. for 3 days in a molar ratio of 1:2. After cooling, the mixture was centrifuged at 5000 rpm for 5 minutes, then the residue was washed 3 times with degassed water and finally dried at room temperature.

When using either the sodium salts of alendronate or risedronate near complete intercalation resulted, which approached about 3.7 meq/gram.

Generalized Matrix Tablet

A matrix tablet is a compressed dosage form containing the appropriate bisphosphonate (from about 70-150 mg bisphosphonate)-hydrotalcite clay, matrix agent, plus fillers, lubricants and excipients. Using water-soluble Methocel F as a rate-controlling polymer, the matrix may be tableted by direct compression or conventional wet granulation. Water permeates into the tablet causing the gel layer to become thicker. Soluble drug diffuse out of the gel layer at a rate controlled by the gel viscosity. With soluble drugs, the primary release mechanism is by diffusion through the gel layer. The amount of Methocel F in the matrix tablet is about 25 wt. percent.

Formulated Tablets (Using Alendronate Mono-Sodium Trihydrate)

### EXAMPLE 1

| | |
|---|---|
| Alendronate - hydrotalcite | 250 mg |
| Corn starch | 25 mg |
| Magnesium stearate | 10 mg |
| Methocel F | 85 mg |
| Lactose | 100 mg |

### EXAMPLE 2

| | |
|---|---|
| Alendronate - hydrotalcite | 200 mg |
| Lactose | 25 mg |
| Allodial silica | 5 mg |
| Corn starch | 30 mg |
| Methocel F | 70 mg |
| Magnesium stearate | 5 mg |

Overall this invention advances the therapeutic treatment of osteoporosis by disclosing a new control release system with biological bisphosphonate and optionally include other synergistic additives.
Advantages Include:
Control release
Improved bioavailability
Reduced irritation
Mask bitter taste
Incorporate analgesic agents, amine narcotics, vitamins or pro-vitamins, hormones, and/or neutraceuticals

1. A pharmaceutical composition or admixture comprising a bioactive bisphosphonate and intercalated with an inorganic layered host, and optionally having an enteric coating consisting of a water-soluble or hydrocolloid polymer and excipients like fillers and lubricants.

2. The drug of claim 1, wherein the bioactive bisphosphonate is selected from the group consisting of pamidronate, minodronate, ibandronic, risedronate, cimadronate, clodronate, neridronate, olpadronate, piridronate, teludronate, zolendronate, icadronate, alendronate or etidronate having at least one alkali or alkaline atom as a salt of a phosphonic acid.

3. The layered inorganic host of claim 1 consisting of a hydrotalcite or hydrotalcite-like mineral with exchangeable anions.

4. The layered inorganic host of claim 1 consisting of a hydroxy double salts of the general formula (M²⁺)₅(OH)₈.(Aⁿ⁻)_{2/n}.yH₂O or (M²⁺)₂(OH)₃.(Aⁿ⁻)_{1/n}.yH₂O with exchangeable anions.

5. The layered inorganic host of claim 3 wherein the binding capacity to a bisphosphonate anion ranges from about 1.0 meq/1 gram to about 3.9 meq/gram.

6. The layered inorganic host of claim 4 wherein the binding capacity to a bisphosphonate anion ranges from about 1.0 meg/gram to about 3.6 meg/gram.

7. The layered inorganic host consisting as an admixture with a bioactive phosphonate as described in claim 1.

8. The layered inorganic host of claim 7 comprising a hydrotalcite mineral.

9. The layered inorganic host of claim 7 comprising a hydroxy double salt of the following formula
(M²⁺)₅(OH)₈.(Aⁿ⁻)_{2/n}.yH₂O or (M²⁺)₂(OH)₃.(Aⁿ⁻¹)_{1/n}.yH₂O

10. The pharmaceutical composition or admixture as described in claim 1 whereby a synergistic bioactive molecule in it's anionic form is intercalated into a hydrotalcite or hydrotalcite clay.

11. The pharmaceutical composition or admixture as described in claim 10 whereby the bioactive anionic molecule is an non-steroidal anti-inflammatory drug.

12. The pharmaceutical composition or admixture as described in claim 11 whereby the non-steroidal anti-inflammatory drug can be aspirin, ibuprofren, diclofenac, diflunisal, etodolac, fenoprofen, flurbiprofen, indomethacin, ketoprofen, ketofolac, mefenamic acid, naproxen, oxaprozin, piroxicam, salsalate, sulindac, tolmetin and the like.

13. The pharmaceutical composition or admixture as described in claim 10 whereby the bioactive anionic molecule is a vitamin or pro-vitamin.

14. The pharmaceutical composition or admixture as described in claim 13 whereby the vitamin or pro-vitamins is vitamin C and/or vitamin E.

15. The pharmaceutical compositions or admixture hydrotalcite or hydrotalcite-like clay of claims 4 and 9, whereby the exchangeable anion is phosphate, polyphosphate, or pyrophosphate.

16. The pharmaceutical compositions or admixture of claim 1 whereby sodium phosphate is added in an effective amount to negate any free Al⁺³ ions from forming.

17. The pharmaceutical composition or admixture of claim 1 whereby a chelating agent is the anionic molecule intercalated.

18. The chelating agent as described in claim 17 is a amino carboxylate, or a amino phosphonate molecule.
